(19) United States
(12) Patent Application Publication
(10) Pub. No.: US 2020/0060534 A1
(43) Pub. Date: Feb. 27, 2020

(54) ENDOSCOPE

(71) Applicant: FUJIFILM Corporation, Tokyo (JP)

(72) Inventors: Hiroaki TABE, Kanagawa (JP); Yoshinori MORIMOTO, Kanagawa (JP)

(73) Assignee: FUJIFILM Corporation, Tokyo (JP)

(21) Appl. No.: 16/510,988

(22) Filed: Jul. 15, 2019

(30) Foreign Application Priority Data
Aug. 23, 2018 (JP) 2018-156076

(51) Int. Cl. A61B 1/07

(52) U.S. Cl. CPC A61B 1/07

(57) ABSTRACT

There are provided an endoscope that has sufficient air-tightness and excellent durability and an endoscope in which a reduction in the amount of light is suppressed.

An endoscope includes: a light guide that emits incident light from an end portion thereof; a distal end member that is provided at the end portion of the light guide, has a function to propagate the light emitted from the light guide by total reflection, and propagates the light emitted from the light guide and emits the light from a distal end face thereof; and an imaging unit that uses the light emitted from the distal end member to image an object to be observed. The distal end member includes a light guide member made of glass.

### CROSS-REFERENCE TO RELATED APPLICATIONS

The present application claims priority under 35 U.S.C. § 119 to Japanese Patent Application No. 2018-156076, filed on Aug. 23, 2018. The above application is hereby expressly incorporated by reference, in its entirety, into the present application.

### BACKGROUND OF THE INVENTION

### 1. Field of the Invention

The present invention relates to an endoscope that acquires the image of an object to be observed, and more particularly, to an endoscope including distal end members that are provided at a distal end thereof and have a function to propagate light by total reflection.

### 2. Description of the Related Art

A diagnosis and the like using an endoscope system, which comprises a light source device for an endoscope, an endoscope, and a processor device, have been widely made in a recent medical care. The light source device for an endoscope generates illumination light, and an object to be observed is irradiated with illumination light from the endoscope.

Lamp light sources, such as a halogen lamp and a xenon lamp emitting white light as the illumination light, have been used as the light source device for an endoscope. However, in recent years, a semiconductor light source, such as a laser diode (LD) or a light emitting diode (LED) emitting light having a specific color, is used instead of the lamp light source.

An optical member, such as a cover glass, an imaging element, an imaging lens, and light guides for illumination light arranged around the imaging lens are provided at the distal end of an insertion part of the endoscope.

For example, JP1996-110484A (JP-H08-110484A) discloses an endoscope. Some fibers of an emission end portion of a light-guide-fiber bundle are arranged in illumination windows so as to be tilted in the forward direction of the observation window at a distal end portion of the endoscope where an observation window and the illumination windows are arranged side by side on the front end face of a distal-end-portion body provided at a distal end of an insertion part, and the endoscope includes a distal end portion where an emission end face of the light-guide-fiber bundle is polished along the front end face of the distal-end-portion body.

### SUMMARY OF THE INVENTION

In the endoscope disclosed in JP1996-110484A (JP-H08-110484A), the emission end face of the light-guide-fiber bundle is exposed.

Sterilization treatment using an autoclave, which sterilizes the endoscope while pressurizing and depressurizing the endoscope, is performed on the endoscope by a high-pressure steam sterilizer. However, even though a small gap is present in the endoscope, there is a concern that steam may enter the endoscope through the gap.

Since the light-guide-fiber bundle is fixed by an adhesive applied to the periphery thereof in a structure where the light-guide-fiber bundle is exposed as in JP1996-110484A (JP-H08-110484A), there is a concern that steam may enter the endoscope from an interface. For this reason, there is a problem that air-tightness is not sufficient and durability is poor.

Further, in a case where the endoscope is used, dirt is not sufficiently removed even though cleaning is performed. For this reason, the amount of illumination light may be reduced. The amount of illumination light may be reduced in the structure where the light-guide-fiber bundle is exposed as described above.

An object of the invention is to solve problems based on the above-mentioned related art and to provide an endoscope that has sufficient air-tightness and excellent durability.

Another object of the invention is to provide an endoscope in which a reduction in the amount of light is suppressed.

To achieve the above-mentioned objects, the invention provides an endoscope comprising: a light guide that emits incident light from an end portion thereof; a distal end member that is provided at the end portion of the light guide, has a function to propagate the light emitted from the light guide by total reflection, and propagates the light emitted from the light guide and emits the light from a distal end face thereof; and an imaging unit that uses the light emitted from the distal end member to image an object to be observed. The distal end member comprises a light guide member made of glass.

It is preferable that the light guide and the distal end member are formed integrally.

The invention provides an endoscope comprising: a light guide that emits incident light from an end portion thereof; a distal end member that is provided at the end portion of the light guide, has a function to propagate the light emitted from the light guide by total reflection, and propagates the light emitted from the light guide and emits the light from a distal end face thereof; and an imaging unit that uses the light emitted from the distal end member to image an object to be observed. The light guide and the distal end member are formed integrally, and the distal end member comprises a light guide member made of glass.

It is preferable that the distal end member includes a core and a clad of which a refractive index is lower than a refractive index of the core and the core has a size covering the end portion of the light guide.

It is preferable that the core is made of glass and the clad is provided on an entire side surface of the core.

It is preferable that a numerical aperture of the distal end member is equal to or larger than a numerical aperture of the light guide.

It is preferable that the distal end member is formed of an optical fiber bundle where a plurality of optical fibers are bundled by fusion welding and the distal end member includes an end face covering the end portion of the light guide.

It is preferable that the optical fiber includes a core and a clad and a numerical aperture of the optical fiber is equal to or larger than a numerical aperture of the light guide.

It is preferable that the distal end member includes a coating material disposed on an outer peripheral surface of the optical fiber bundle and having a refractive index equal to or lower than a refractive index of the clad.

It is preferable that the endoscope further includes a holding tube in which the imaging unit is provided and the distal end member is fixed to an outer periphery of the holding tube by brazing.

It is preferable that the light guide is fixed to the outer periphery of the holding tube.

According to the invention, it is possible to provide an endoscope that has sufficient air-tightness and excellent durability. In addition, according to the invention, it is possible to provide an endoscope in which a reduction in the amount of light is suppressed.

### BRIEF DESCRIPTION OF THE DRAWINGS

FIG. 1 is a schematic diagram showing an example of an endoscope system that includes an endoscope according to an embodiment of the invention.

FIG. 2 is a schematic plan view showing an insertion-side distal end face of the endoscope according to the embodiment of the invention.

FIG. 3 is a schematic cross-sectional view of an insertion-side distal end portion of the endoscope according to the embodiment of the invention.

FIG. 4 is a schematic side view showing a first example of a light guide member of the endoscope according to the embodiment of the invention.

FIG. 5 is a schematic plan view showing the first example of the light guide member of the endoscope according to the embodiment of the invention.

FIG. 6 is a partial enlarged view showing the first example of the light guide member of the endoscope according to the embodiment of the invention.

FIG. 7 is a schematic side view showing a second example of the light guide member of the endoscope according to the embodiment of the invention.

FIG. 8 is a schematic plan view showing the second example of the light guide member of the endoscope according to the embodiment of the invention.

FIG. 9 is a partial enlarged view showing the second example of the light guide member of the endoscope according to the embodiment of the invention.

FIG. 10 is a schematic diagram showing an example of a surgical apparatus including the endoscope according to the embodiment of the invention.

### DESCRIPTION OF THE PREFERRED EMBODIMENTS

An endoscope of the invention will be described in detail below on the basis of preferred embodiments shown in the accompanying drawings.

Drawings to be described below are illustrative to describe the invention, and the invention is not limited to the drawings to be described below.

“to” showing a numerical range in the following description means that numerical values written on both sides of the numerical range are included. For example, a case where ε is in the range of a numerical value α to a numerical value β means that the range of s is a range including the numerical value α and the numerical value β, and is represented as “α≤ε≤β” by mathematical symbols.

Further, “overall” and the like include the range of an error that is generally allowed in a corresponding technical field.

Endoscope System

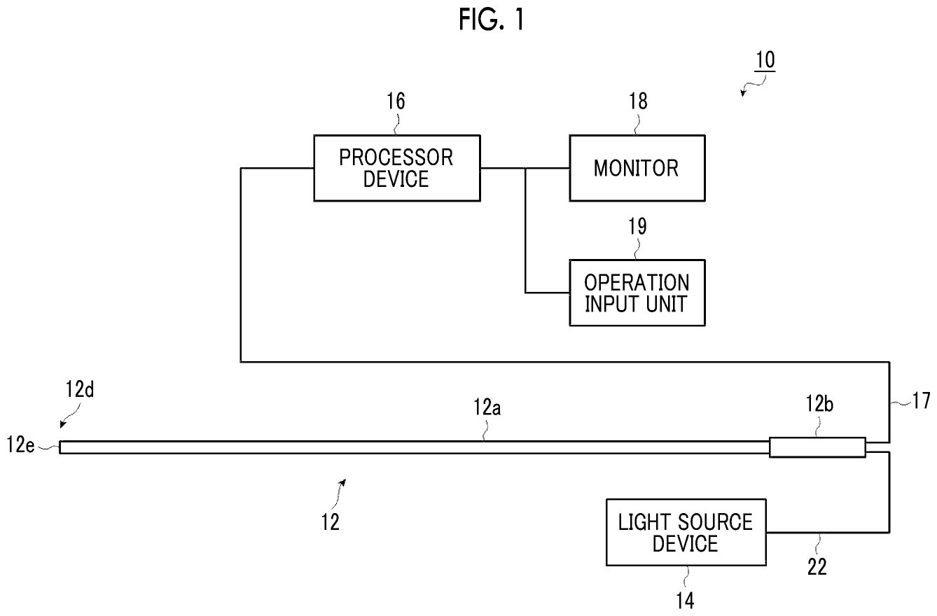

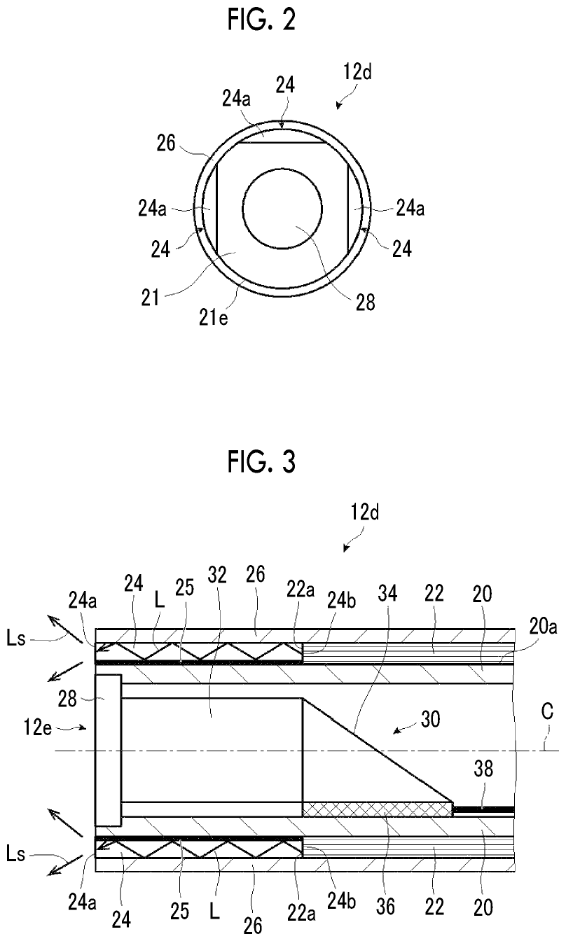

FIG. 1 is a schematic diagram showing an example of an endoscope system that includes an endoscope according to an embodiment of the invention, FIG. 2 is a schematic plan view showing an insertion-side distal end face of the endoscope according to the embodiment of the invention, and FIG. 3 is a schematic cross-sectional view of an insertion-side distal end portion of the endoscope according to the embodiment of the invention.

An endoscope system 10 irradiates a portion to be observed present in a living body (in a subject), which is an object to be observed, with illumination light Ls (see FIG. 3), images the portion to be observed, generates a display image of the portion to be observed on the basis of image signals obtained from imaging, and displays the display image.

As shown in FIG. 1, the endoscope system 10 comprises an endoscope 12 that images a portion to be observed present in a living body (in a subject), which is an object to be observed, a processor device 16 that generates a display image of the portion to be observed on the basis of image signals obtained from imaging, a light source device 14 for an endoscope (hereinafter, simply referred to as a light source device) that supplies illumination light Ls (see FIG. 3) illuminating the portion to be observed to the endoscope 12, and a monitor 18 that displays the display image. An operation input unit 19, such as a keyboard and a mouse, is connected to the processor device 16.

The endoscope 12 comprises an insertion part 12a that is to be inserted into a subject, such as a patient's body, and an operation unit 12b that is provided at the proximal end portion of the insertion part 12a. A side corresponding to the insertion part 12a is the distal end side of the endoscope 12. The operation unit 12b of the endoscope 12 and the processor device 16 are connected to each other by a signal line 17. For example, the endoscope 12 is a direct-view type rigid endoscope, such as a laparoscope.

The processor device 16 receives image signals, which are output from an imaging unit 30 (see FIG. 3) of the endoscope 12, through the signal line 17, generates video signals, and outputs the video signals to the monitor 18. Accordingly, the display image of the portion to be observed present in a body or the like is projected on the monitor 18.

The operation unit 12b of the endoscope 12 and the light source device 14 are connected to each other by a light guide 22. Light generated from the light source device 14 is supplied to the light guide 22, so that light is emitted from a distal end face 12e of the endoscope 12.

As shown in FIG. 2, a disc-shaped cover glass 28 is disposed on the distal end face 12e of a distal end portion 12d of the endoscope 12. For example, three distal end members 24 are arranged so as to surround the cover glass 28. Each distal end member 24 has a function to propagate light, which is emitted from the light guide 22, by total reflection. The distal end member 24 propagates light L (see FIG. 3), which is emitted from the light guide 22, and emits illumination light Ls (see FIG. 3) from a front end face 24a. A protective member 21 is provided between the cover glass 28 and the distal end members 24. The protective member 21 is made of, for example, steel special use stainless (SUS).

For example, as shown in FIG. 2, a ratio of an area, which is occupied by the distal end members 24, to the area of the distal end face 12e of the endoscope 12 is lower than a ratio of an area, which is occupied by the cover glass 28, to the area of the distal end face 12e. For this reason, since the endoscope has relatively high resistance against an impact caused by a fall, the durability of the endoscope 12 is improved.

Positions where the distal end members 24 are arranged and the number of the distal end members 24 are not particularly limited. One distal end member 24 may be formed to surround the entire circumference of the cover glass 28, or a plurality of distal end members 24 may be formed to surround the entire circumference of the cover glass 28.

As shown in FIG. 3, the endoscope 12 includes a hollow cylindrical holding tube 20 and a hollow cylindrical outer tube 26, and the holding tube 20 is housed in the outer tube 26. The outer periphery of the outer tube 26 is the outer periphery of the insertion part 12a of the endoscope 12. The outer tube 26 is made of, for example, ceramics.

The light guide 22 and the distal end members 24 are disposed between the holding tube 20 and the outer tube 26. All the light guide 22 and the distal end members 24 are provided in the insertion part 12a of the endoscope 12, that is, at the distal end of the endoscope 12.

The distal end members 24 are provided in front of the side of the light guide 22 from which light L is to be emitted. Rear end faces 24b of the distal end members 24 are in contact with an end face 22a of the light guide 22 from which light L is to be emitted. The light guide 22 is fixed to an outer periphery 20a of the holding tube 20 by, for example, an adhesive. Since the distal end members 24 are fixed to the outer periphery 20a of the holding tube 20 by, for example, brazing although described later, the distal end members 24 are fixed to the outer periphery of the holding tube 20 through a bonding layer 25.

The light guide 22 and the distal end members 24 are formed separately, but are not limited thereto. The light guide 22 and the distal end members 24 may be formed integrally.

The disc-shaped cover glass 28 is provided in the opening of the holding tube 20, and the holding tube 20 is airtightly sealed by the cover glass 28. Accordingly, the inside of the holding tube 20 is kept in an airtight state.

It is preferable that a metallized layer (not shown) is formed on the side surface of the cover glass 28 and a plating layer is also formed on the fixing surface of the holding tube 20 to which the cover glass 28 is to be fixed. Accordingly, the cover glass 28 can be bonded to the holding tube 20 with high air-tightness by solder.

The cover glass 28 is not particularly limited.

The imaging unit 30 is provided in the holding tube 20. The imaging unit 30 uses light, which is emitted from the distal end members 24, to image an object to be observed. The imaging unit 30 includes, for example, a lens unit 32, a prism 34, and an imaging element 36. The lens unit 32 is provided to be in contact with the cover glass 28. The prism 34 is, for example, a rectangular prism.

The lens unit 32 is provided to be in contact with one surface of the surfaces of the prism 34 that cross each other at a right angle. The imaging element 36 is provided to be in contact with the other surface of the surfaces of the prism 34 that cross each other at a right angle.

The prism 34 leads the optical path of light of the lens unit 32 to the imaging element 36, and may not be needed according to a position where the imaging element 36 is disposed. Further, since a prism suitable for a position where the imaging element 36 is disposed is appropriately used as the prism 34, the prism 34 is also not limited to the rectangular prism in terms of a shape.

A signal line 38 is electrically connected to the imaging element 36. The signal line 38 transmits image signals, which are to be output from the imaging element 36, and is electrically connected to the above-mentioned signal line 17 (see FIG. 1). The signal line 17 is connected to the processor device 16 as described above.

A charge coupled device (CCD) image sensor or a complementary metal oxide semiconductor (CMOS) image sensor can be used as the imaging element 36.

The lens unit 32 includes, for example, a lens barrel (not shown) and lenses (not shown) that are provided in the lens barrel. The number of the lenses is not particularly limited, and a plurality of lenses may be arranged in parallel in the direction of an optical axis C or only one lens may be provided.

The light guide 22 is a light transmission member, and is formed of, for example, a bundle of a plurality of optical fiber strands (not shown). Light generated from the light source device 14 (see FIG. 1) is supplied to the light guide 22 as described above, light L emitted from the end face 22a of the light guide 22 is incident on the rear end faces 24b of the distal end members 24, and illumination light Ls is emitted to a portion to be observed from the front end faces 24a of the distal end members 24.

The endoscope 12 irradiates an affected part with, for example, light having an angle of aperture of 120° or more from the front end faces 24a of the distal end members 24. For example, in a surgical operation using the endoscope 12, an operator images the affected part, which is irradiated with light, through the cover glass 28 by the imaging element 36, and operates a treatment tool (not shown) to treat the affected part while confirming the video of the affected part on the monitor 18.

The distal end members 24 will be described below.

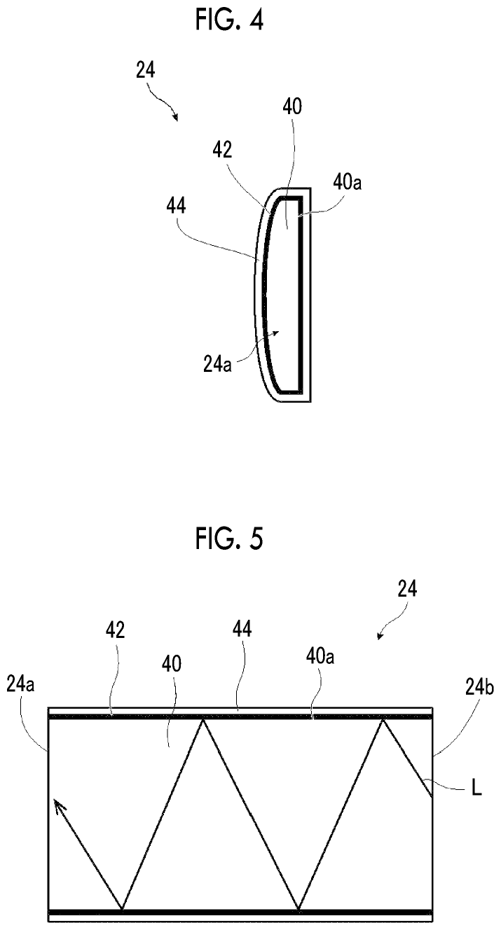

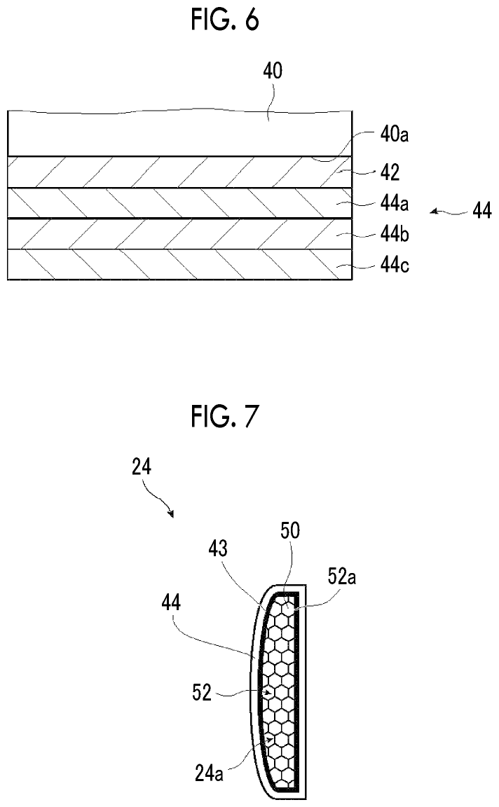

FIG. 4 is a schematic side view showing a first example of a light guide member of the endoscope according to the embodiment of the invention, FIG. 5 is a schematic plan view showing the first example of the light guide member of the endoscope according to the embodiment of the invention, and FIG. 6 is a partial enlarged view showing the first example of the light guide member of the endoscope according to the embodiment of the invention. FIG. 4 shows the shape of the distal end member 24 that is viewed from the front end face 24a.

Each distal end member 24 has a function to propagate light, which is emitted from the light guide 22, by total reflection as described above. The distal end member 24 includes, for example, a core 40 and a clad 42 as shown in FIGS. 4 and 5. The clad 42 is provided on the entire side surface 40a of the core 40. The core 40 has a size covering the end portion of the light guide 22, and the end face 22a of the light guide 22 is covered with the cores 40. Accordingly, the light L (see FIG. 3), which is emitted from the light guide 22, is incident on the rear end faces 24b of the distal end members 24.

The refractive index of the clad 42 is lower than the refractive index of the core 40. Further, a protective film 44 is provided on the clad 42. The protective film 44 is a film that is required to fix the distal end member 24 to the holding tube 20. A refractive index is a value that is measured by a minimum deviation method.

For example, the core 40 of the distal end member 24 is made of glass. For example, S-LAL14 (nitrate seed) manufactured by Ohara Corporation is used as the glass that is used to make the core 40.

The clad 42 is made of silicon dioxide (SiO₂). The clad 42 is a silicon dioxide (SiO₂) layer. The light L, which is emitted from the light guide 22 and is incident on the rear end face 24b of the distal end member 24, is propagated by total reflection using a difference between the refractive index of the glass, which is the material of the core 40, and the refractive index of SiO₂, which is the material of the clad 42, and is emitted from the front end face 24a of the distal end member 24 as the illumination light Ls (see FIG. 3).

The distal end member 24 includes a light guide member that is made of glass like the above-mentioned core 40. The clad 42 may also be made of magnesium fluoride other than silicon dioxide (SiO₂).

In a case where the refractive index of the core 40 is denoted by n₁ and the refractive index of the clad 42 is denoted by n₂, the numerical aperture NA of the distal end member 24 is represented by the following equation. It is preferable that the refractive index n₁ of the core 40 is 1.695 (nD) or more. Further, it is preferable that the refractive index n₂ of the clad 42 is 1.46 (nD) or less. The distal end member 24 functions as an optical fiber of which NA is larger than 0.86. D of the above-mentioned nD means that a measurement wavelength is 589 nm (sodium D line).

The refractive index of the core 40 and the refractive index of the clad 42 are values that are measured by a minimum deviation method as described above.

A numerical aperture is an index representing which light can be collected. The maximum acceptance angle is larger as the numerical aperture is larger. Since light is more likely to be collected as a numerical aperture is larger as described above, it is preferable that the numerical aperture NA of the distal end member 24 is equal to or larger than the numerical aperture Nag of the light guide 22. That is, it is preferable that “NA≥NAg” is satisfied.

NA=\sqrt{n₁²−n₂²} Expression 1

Further, the protective film 44 is, for example, a film having a multilayer structure where a Cr film 44a, a Ni film 44b, and an Au film 44c are stacked in this order from the clad 42.

The Cr film 44a and the Ni film 44b are films that are required for the adhesion of the Au film 44c and the protection of the core 40. The Au film 44c is a film that is required to fix the distal end member 24 to the outer periphery 20a of the holding tube 20 by brazing. Since the distal end member 24 is fixed to the outer periphery 20a of the holding tube 20 by brazing, air-tightness can be improved.

The clad 42, the Cr film 44a, the Ni film 44b, and the Au film 44c are formed by, for example, a sputtering method.

As long as the above-mentioned function can be fulfilled, the composition of the films and the number of the films are not particularly limited in terms of the structure of the protective film 44.

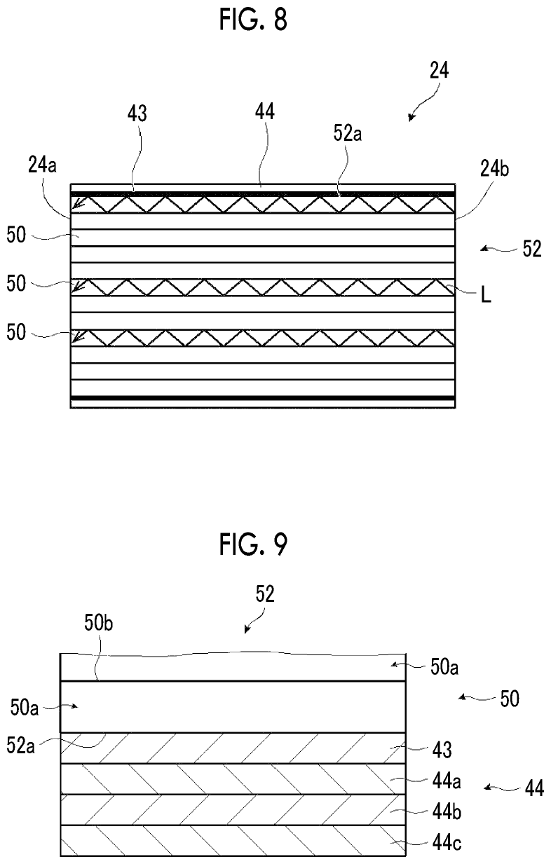

FIG. 7 is a schematic side view showing a second example of the light guide member of the endoscope according to the embodiment of the invention, FIG. 8 is a schematic plan view showing the second example of the light guide member of the endoscope according to the embodiment of the invention, and FIG. 9 is a partial enlarged view showing the second example of the light guide member of the endoscope according to the embodiment of the invention. FIG. 7 shows the shape of the distal end member 24 that is viewed from the front end face 24a.

The same components as the components of the distal end member 24 shown in FIGS. 4 to 6 are denoted in FIGS. 7 to 9 by the same reference numerals as the reference numerals shown in FIGS. 4 to 6, and the detailed description thereof will be omitted.

A distal end member 24 shown in FIGS. 7 and 8 and the distal end member 24 shown in FIGS. 4 and 5 have the same shape.

The distal end member 24 shown in FIGS. 7 and 8 is formed of an optical fiber bundle 52 that is a bundle of a plurality of optical fibers 50. The optical fiber 50 includes a core 50a and a clad 50b, and is made of, for example, glass.

It is preferable that the refractive index n₁ of the core 50a is 1.65 (nD) or more. Further, it is preferable that the refractive index n₂ of the clad 50b is in the range of 1.3 to 1.5 (nD).

Light can be propagated by total reflection in the optical fiber bundle 52 as in the original optical fibers 50 that are not yet subjected to thermal fusion welding. Even in the case of the distal end member 24 formed of the optical fiber bundle 52, light L, which is emitted from the light guide 22 and is incident on the rear end face 24b of the distal end member 24, is propagated by total reflection in each optical fiber 50 and is emitted from the front end face 24a of the distal end member 24 as illumination light Ls (see FIG. 3). The distal end member 24 includes a light guide member that is made of glass like the above-mentioned optical fiber 50.

The optical fiber bundle 52 is a bundle where the plurality of optical fibers 50 are fixed and bundled in the form of glass by thermal fusion welding. The optical fiber bundle 52 includes an end face that covers the end portion of the light guide 22. This end face is the rear end face 24b of the distal end member 24. Accordingly, the light emitted from the light guide 22 is incident on the rear end faces 24b of the distal end members 24.

A coating material is provided on an outer peripheral surface 52a of the optical fiber bundle 52 instead of the clad 42, so that a coating layer 43 is provided on the outer peripheral surface 52a. A protective film 44 is provided on the coating layer 43.

The coating material has a refractive index equal to or lower than the refractive index of the clad 50b of the optical fiber 50. The coating layer 43 fulfills the same function as the clad 42 shown in FIGS. 4 to 6. The coating material is made of, for example, silicon dioxide (SiO₂) or magnesium fluoride like the clad 42.

Since the coating layer 43 is provided, light can be totally reflected by the coating layer 43 even though some of the optical fibers 50 of the optical fiber bundle 52 are absent. Accordingly, light can be propagated by total reflection.

Further, since light is more likely to be collected as a numerical aperture is larger, it is preferable that the numerical aperture of the optical fiber 50 is equal to or larger than the numerical aperture of the light guide 22 even in the case of the optical fiber bundle 52.

Since the endoscope 12 is provided with the distal end members 24 comprising the light guide members made of glass as described above, the light guide 22 is not exposed to the distal end face 12e of the endoscope 12. For this reason, the light guide 22 is protected even in a case where, for example, sterilization treatment is performed by an autoclave. Accordingly, durability can be improved.

Further, since the distal end members 24 are provided, high air-tightness can be maintained. Accordingly, even in a case where, for example, sterilization treatment using an autoclave is performed, the ingress of steam is suppressed. This can also cause durability to be improved.

Furthermore, since the light guide 22 is not exposed to the distal end face 12e of the endoscope 12 as described above and the distal end members 24 are exposed, a reduction in the amount of illumination light caused by dirt can be suppressed and a reduction in the amount of illumination light caused by degradation over time can also be suppressed.

In a case where the entire distal end face 12e of the endoscope 12 is covered with the cover glass 28 or the like, illumination light is obstructed if the cover glass 28 is thick. Accordingly, the amount of illumination light is reduced. For this reason, the cover glass 28 needs to be made thin and the durability of the cover glass 28 is lowered as much as that. However, a problem, which occurs in a case where the entire distal end face 12e of the endoscope 12 is covered with the above-mentioned cover glass 28, is not caused in the case of a structure where the distal end members 24 are provided.

Further, in a structure where the light guide and the imaging unit are covered with one cover glass, light subjected to Fresnel reflection on the surface of the cover glass may be incident on the imaging element. A function to prevent return light, which is realized by a polarizer, a wave plate, and the like, needs to be provided to prevent this, but not only an increase in cost is caused but also a reduction in the amount of light is also caused. In contrast, since the distal end members 24 are provided and the cover glass of the imaging unit is provided separately, the problem of the structure where the light guide and the imaging unit are covered with one cover glass, is not caused.

A rigid endoscope has been exemplified as the above-mentioned endoscope 12. However, the endoscope 12 is not limited to the rigid endoscope, and the invention can also be applied to a flexible endoscope of which an insertion part includes a flexible portion, a bendable portion, and a distal end-rigid portion. The use of the endoscope 12 is not limited to the above-mentioned endoscope system 10 shown in FIG. 1. A use example of the endoscope 12 will be described below.

Use Example of Endoscope 12

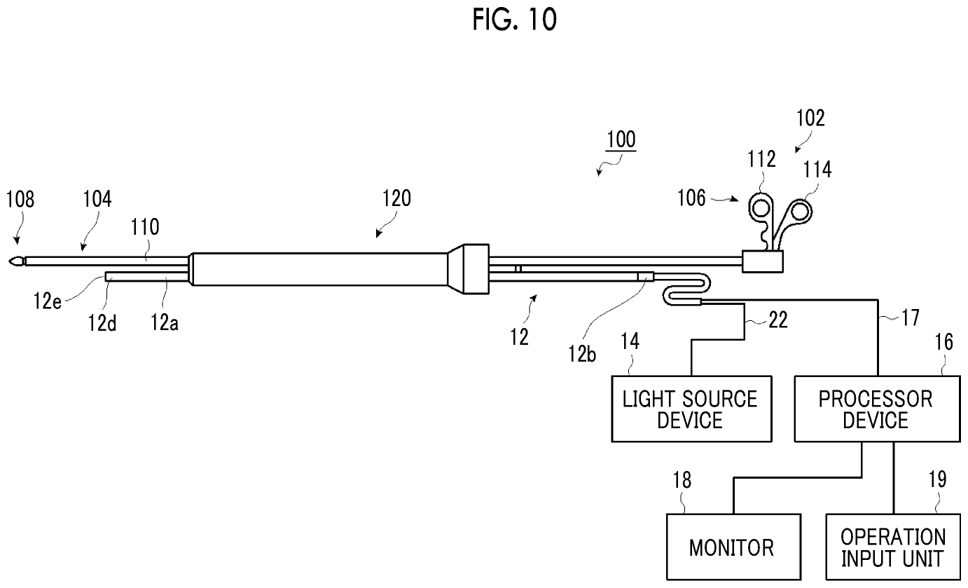

FIG. 10 is a schematic diagram showing an example of a surgical apparatus including the endoscope according to the embodiment of the invention. FIG. 10 shows an example where the endoscope 12 is applied to an endoscopic surgical apparatus 100.

The endoscopic surgical apparatus 100 includes an endoscope 12 that is used to observe the inside of a patient's body cavity, a treatment tool 102 that is used to examine or treat an affected part in the patient's body cavity, and a sheath tube 120 that guides the endoscope 12 and the treatment tool 102 into the body cavity.

The treatment tool 102 comprises: an elongated treatment tool-insertion part 104 that is formed of, for example, a forceps and is to be inserted into a body cavity; an operation unit 106 that is provided on the proximal end side of the treatment tool-insertion part 104 and is to be gripped by an operator; and a treatment part 108 that is provided on the distal end side of the treatment tool-insertion part 104 and can be actuated by the operation of the operation unit 106.

The sheath tube 120 includes an endoscope-insertion passage (not shown) into which the insertion part 12a of the endoscope 12 is inserted to be movable back and forth, and a treatment tool-insertion passage (not shown) into which the treatment tool-insertion part 104 of the treatment tool 102 is inserted to be movable back and forth.

The treatment tool-insertion part 104 is provided with a cylindrical sheath 110 and an operation shaft (not shown) that is inserted into the sheath 110 to be movable in an axial direction. The operation unit 106 is further provided with a stationary handle 112 and a movable handle 114 that is connected to the stationary handle 112 through a rotational movement pin so as to be movable rotationally relative to the stationary handle 112. Further, a proximal end portion of the operation shaft is connected to the movable handle 114.

The treatment part 108 is provided with a pair of grip members that can be opened and closed. These grip members are connected to the distal end portion of the operation shaft through a drive mechanism. The grip members of the treatment part 108 are opened and closed through the operation shaft and the drive mechanism with the rotational movement operation of the movable handle 114 of the operation unit 106.

The treatment tool 102 is not limited to a forceps, and may be, for example, other treatment tools, such as a laser probe, suture instruments, an electric knife, a needle forceps, and an ultrasonic aspirator.

The invention basically has the above-mentioned structure. The endoscope according to the embodiment of the invention has been described in detail above, but it is natural that the invention is not limited to the above-mentioned embodiment and the invention may have various improvements and modifications without departing from the scope of the invention.

### EXPLANATION OF REFERENCES

10: endoscope system
12: endoscope
12a: insertion part
12b: operation unit
12d: distal end portion
12e: distal end face
14: light source device for endoscope (light source device)
16: processor device
17: signal line
18: monitor
19: operation input unit
20: holding tube
20a: outer periphery
21: protective member
22: light guide
22a: end face
24: distal end member
24a: front end face
24b: rear end face
26: outer tube
28: cover glass
30: imaging unit
32: lens unit
34: prism
36: imaging element
38: signal line
40, 50a: core
40a: side surface
42, 50b: clad
43: coating layer
44: protective film
44a: Cr film
44b: Ni film
44c: Au film
50: optical fiber
52: optical fiber bundle
52a: outer peripheral surface
100: endoscopic surgical apparatus
102: treatment tool
104: treatment tool-insertion part
106: operation unit
108: treatment part
110: sheath
112: stationary handle
114: movable handle
120: sheath tube
C: optical axis
L: light
Ls: illumination light

What is claimed is:

1. An endoscope comprising:
a light guide that emits incident light from an end portion thereof;
a distal end member that is provided at the end portion of the light guide, has a function to propagate the light emitted from the light guide by total reflection, and propagates the light emitted from the light guide and emits the light from a distal end face thereof; and
an imaging unit that uses the light emitted from the distal end member to image an object to be observed,
wherein the distal end member comprises a light guide member made of glass.

2. The endoscope according to claim 1,
wherein the light guide and the distal end member are formed integrally.

3. An endoscope comprising:
a light guide that emits incident light from an end portion thereof;
a distal end member that is provided at the end portion of the light guide, has a function to propagate the light emitted from the light guide by total reflection, and propagates the light emitted from the light guide and emits the light from a distal end face thereof; and
an imaging unit that uses the light emitted from the distal end member to image an object to be observed,
wherein the light guide and the distal end member are formed integrally, and
the distal end member comprises a light guide member made of glass.

4. The endoscope according to claim 1,
wherein the distal end member includes a core and a clad of which a refractive index is lower than a refractive index of the core, and
the core has a size covering the end portion of the light guide.

5. The endoscope according to claim 2,
wherein the distal end member includes a core and a clad of which a refractive index is lower than a refractive index of the core, and
the core has a size covering the end portion of the light guide.

6. The endoscope according to claim 3,
wherein the distal end member includes a core and a clad of which a refractive index is lower than a refractive index of the core, and
the core has a size covering the end portion of the light guide.

7. The endoscope according to claim 4,
wherein the core is made of glass and the clad is provided on an entire side surface of the core.

8. The endoscope according to claim 6,
wherein the core is made of glass and the clad is provided on an entire side surface of the core.

9. The endoscope according to claim 4,
wherein a numerical aperture of the distal end member is equal to or larger than a numerical aperture of the light guide.

10. The endoscope according to claim 6,
wherein a numerical aperture of the distal end member is equal to or larger than a numerical aperture of the light guide.

11. The endoscope according to claim 1,
wherein the distal end member is formed of an optical fiber bundle where a plurality of optical fibers are bundled by fusion welding, and
the distal end member includes an end face covering the end portion of the light guide.

12. The endoscope according to claim 2,
wherein the distal end member is formed of an optical fiber bundle where a plurality of optical fibers are bundled by fusion welding, and
the distal end member includes an end face covering the end portion of the light guide.

13. The endoscope according to claim 3,
wherein the distal end member is formed of an optical fiber bundle where a plurality of optical fibers are bundled by fusion welding, and
the distal end member includes an end face covering the end portion of the light guide.

14. The endoscope according to claim 11,
wherein the optical fiber includes a core and a clad, and a numerical aperture of the optical fiber is equal to or larger than a numerical aperture of the light guide.

15. The endoscope according to claim 14,
wherein the distal end member includes a coating material disposed on an outer peripheral surface of the optical fiber bundle and having a refractive index equal to or lower than a refractive index of the clad.

16. The endoscope according to claim 1, further comprising:
a holding tube in which the imaging unit is provided,
wherein the distal end member is fixed to an outer periphery of the holding tube by brazing.

17. The endoscope according to claim 2, further comprising:
a holding tube in which the imaging unit is provided,
wherein the distal end member is fixed to an outer periphery of the holding tube by brazing.

18. The endoscope according to claim 3, further comprising:
a holding tube in which the imaging unit is provided,
wherein the distal end member is fixed to an outer periphery of the holding tube by brazing.

19. The endoscope according to claim 16,
wherein the light guide is fixed to the outer periphery of the holding tube.
